(19) United States
(12) Patent Application Publication
(10) Pub. No.: US 2007/0254277 A1
(43) Pub. Date: Nov. 1, 2007

(54) Automated systems for handling specimens for laboratory diagnostics and associating relevant information

(76) Inventors: Larry D. Scrabeck, Rochester, MN (US); Jon J. Blixt, Rochester, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/787,656

(22) Filed: Apr. 17, 2007

Related U.S. Application Data
(60) Provisional application No. 60/793,009, filed on Apr. 17, 2006.

(51) Int. Cl. C12Q 1/00; G06F 19/00

(52) U.S. Cl. 435/4; 702/19

(57) ABSTRACT

An automated sorting system provides the ability to receive specimens for any number of diagnostic test procedures and to selectively transfer specimens to designated ones of a plurality of specimen processors based upon criteria of the laboratory facility such as the particular laboratory to perform the requisite diagnostic test, timing aspects like a lab's hours of operation or delivery schedule, type of specimen (e.g. tissue, blood, serum, and the like) or other factor that may affect specimen throughput efficiency. Automated accessioning comprises the determination of physical attributes of specimens using a specimen processor while reading data provided with a specimen as it is provided to the specimen processor (such data provided by the carrier and/or the vial by codes, ID tags, and the like), recording the determined information in a database of a control system, and comparing the determined information with information from a client diagnostic test order from a same or different database for connecting the specimen and an associated electronic record. Preferably also, the accessioning includes a labeling of the specimen after the connection with an electronic record for associating the specimen and record through the specimen diagnostic test procedure and reporting back to the client.

### REFERENCE TO RELATED APPLICATIONS

This application claims the benefit of U.S. Provisional Patent Application having Ser. No. 60/793,009 filed on Apr. 17, 2006, entitled “Automated Systems for Handling Specimens for Laboratory Diagnostics and Associating Relevant Information,” the entire disclosure of which is incorporated herein by reference for all purposes.

### TECHNICAL FIELD

The present invention is directed to automated systems for handling specimens and related information for testing within a diagnostic laboratory. In particular, the present invention is directed to systems and methods for receiving, handling, sorting, verifying and accessioning biological specimens as such are to be tested in a diagnostic laboratory, and for contemporaneously associating relevant information related to the specimens including client and/or patient information along with specimen requirements and/or laboratory diagnostic testing information.

### BACKGROUND OF THE PRESENT INVENTION

Diagnostic testing of biological specimens with respect to disease management or diagnosis is critical in the health care of patients. Certain diagnostic tests are easily conducted within a healthcare facility, such as a physician's office or hospital and are typically handled according to the protocol of that particular healthcare facility to best provide specimen and information integrity. However, with the development of more and more specific diagnostic test procedures for a greater number of diseases or maladies, many of which are uncommon or rare, labs at such healthcare facilities are increasingly less able to conduct such tests as they lack the knowledge, training and/or instrumentation necessary. The more esoteric the diagnosis, the less likely a healthcare facility will be able to conduct such diagnostic testing on their own.

As such, diagnostic laboratories have emerged to provide such services to clients (e.g. the healthcare facilities including physicians and hospitals) as independent service laboratories or by healthcare facilities or research centers that have invested into the diagnostic technologies and training and that have excess laboratory capacity to perform such diagnostic tests for others. In either case, it becomes paramount to receive the specimens and related information correctly before any testing is to take place so that client information and the specimen are used properly to provide timely and accurate test results. Client information typically includes the healthcare facility or physician, patient information such as name and age information, other identification information such as physician and/or patient numbers, and test information such as type of test by name or number, volume or size of specimen, temperature requirements and shipping requirements. Information of this type is known to be provided by paper, electronically, or in other forms, such as linear of two-dimensional bar coding or other symbols or codes. Moreover, information is often provided in an electronic or paper form along with a specimen while also being provided on a specimen vial, such as by a label with printed information and/or bar coding.

Physical specimens themselves reach such laboratories by some delivery service including the use of private, commercial and governmental delivery services. The test order and related information including the physician or healthcare facility ordering the testing, name or ID number of the patient, and test to be conducted primarily arrives through electronic means by way of a private or public network so that the specimen(s) for testing is matched with the information when received. Less often, such test order and related information is sometimes provided along with the specimen where no corresponding electronic test order exists. The acts of linking the physical specimen with its related information, verifying that required information is complete, and checking that the physical specimen is appropriate for diagnostic testing is considered an accessioning step in the diagnostic testing process. In other words, accessioning is the acceptance of a specimen for diagnostic testing based upon completeness of relevant information and sufficiency of the specimen for the desired test. This is today primarily done as a manual process where operators at workstations receive the physical specimens after unpackaging and inspection for suitability and thereafter link the specimen to an electronic record as is contained within a database of the records received as test orders. If an electronic record does not exist at this point, one is created. Thereafter, the specimen and record are linked with a common identification number or code, which number or code is then labeled onto the specimen vial for use throughout the remainder of the test process. Such identification information usually would also include an identification of the particular lab where the diagnostic test is to be conducted, which information facilitates proper delivery of the specimen. When a diagnostic test is completed, the results are reported to the client in electronic form or otherwise and such results are also electronically connected with the test order record and stored in the same or a different database. Of this process, all of the steps that require handling of the specimen prior to loading the specimen on a test instrument are labor intensive, slow, and manual in mature. In particular, the accessioning step takes the greatest amount of manual intervention.

Automation of limited discrete aspects of this process have been done, such as for receiving and opening of delivered packages and for transferring specimens to an accessioning station. However, with specimens arriving from any number of different locations, by different delivery means and within any number of different type and size vials or other vessels, and the corresponding order information also arriving in many different formats and by different means, accessioning that accommodates such variability is time consuming and labor intensive. In order to utilize automation for aspects of this process, laboratory procedures have been developed based upon the use of specific protocols and formats for submitting and receiving a physical specimen, related information and the test order. Then, when such specific procedure, protocols and format are followed, the accessioning step can be expedited with less time and effort. Such prior art attempts at automation have focused on reducing variability of specimen and information input in order to expedite aspects of the accessioning step and to reduce the time and effort involved by the lab. The result of reducing variability is beneficial to the lab; however, client workload and efforts are increased as they are forced to comply and standardize with set procedures and protocols.

Then, after the accessioning of specimens, the specimen must be directed to and delivered to the proper lab where the diagnostic test is to be conducted. In the case of esoteric diagnostic test procedures, many different technological approaches are relied upon, and, as such, many different labs are set up and utilized with distinctly different instrumentation, knowledge and training requirements. Labs are typically distinct from one another based upon the type of instrumentation and testing that are utilized for the diagnostic testing, including technologies based upon hematology, biochemistry, immunology, and microbiology, for examples. As such, even after the accessioning of specimens, the sorting and delivery of specimens and related records to a proper lab is time and labor intensive.

The ability to conduct esoteric diagnostic testing requires that a laboratory facility include many different labs directed to each of the specialized technological disciplines to provide an alternative to having individual healthcare facilities provide such services themselves. However, to be commercially viable as an esoteric laboratory facility, a large volume of specimens are processed on any given day, which may include many tens or perhaps hundreds of thousands of specimens and diagnostic tests from a very broad and diverse client base. This volume of receiving, sorting, accessioning and testing exacerbates the intensity of time and labor involved, in particular, in these processing steps as discussed above.

For use within a particular lab, lab automation systems have been developed as front-end specimen processors. As specimens are delivered to a lab after accessioning, the specimen vials are positioned within a distribution machine in trays. From such tray, a transfer gripper of the distribution machine that is movable in the X-Y plane picks up one or more specimen vials at a time and transfers them to a conveyor that moves each specimen vial past a decapper device and a reader (to read a bar code, for example). Based upon knowledge of the record accessible by a computer station after specimen identification from the reader, another transfer gripper that is also movable in the X-Y plane moves the specimen vial or vials from the conveyor to a predetermined tray that is designated for the specific diagnostic test desired to be conducted. This process is conducted over and over until all specimens for that lab are sorted to a particular tray for each diagnostic test to be conducted in that lab. Such a system is described in U.S. Pat. Nos. 6,151,535 and 6,220,451.

A particular example of a commercially available distribution machine is the OLA2500 Clinical Lab Automation System from Olympus America Inc. of Melville, N.Y. This system provides the transfer features discussed above and also provides features for determining specimen volume and for automated aliquoting of the specimen into plural secondary tubes or vials. With test procedure information and test prioritization protocols or client directions as stored within a computer accessible database, prioritized aliquoting can be done when insufficient specimen volume is determined in the primary or specimen supply vial. Furthermore, this system includes the ability to produce labels with information or codes and to apply such labels as needed to such secondary tubes.

### SUMMARY OF THE PRESENT INVENTION

The present invention overcomes the disadvantages and shortcomings of the prior art by providing a system including automated components for sorting and accessioning specimens that permits variability in the manner of specimen and information submission to a laboratory facility to perform diagnostic testing.

An automated sorting system provides the ability to receive specimens for any number of diagnostic test procedures and to selectively transfer specimens to designated ones of a plurality of specimen processors based upon criteria of the laboratory facility such as the particular laboratory to perform the requisite diagnostic test, timing aspects like a lab's hours of operation or delivery schedule, type of specimen (e.g. tissue, blood, serum, and the like) or other factor that may affect specimen throughput efficiency.

Automated accessioning comprises the determination of physical attributes of specimens using a specimen processor while reading data provided with a specimen as it is provided to the specimen processor (such data provided by the carrier and/or the vial by codes, ID tags, and the like), recording the determined information in a database of a control system, and comparing the determined information with information from a client diagnostic test order from a same or different database for connecting the specimen and an associated electronic record. Preferably also, the accessioning includes a labeling of the specimen with a unique specimen ID after the connection with an electronic record for associating the specimen and record through the specimen diagnostic test procedure and reporting back to the client.

By automating one or both of the sorting and accessioning steps within a laboratory system, human touch points of specimens can be greatly reduced. This not only facilitates a reduction of labor intensity, but also reduces the possibility of specimens being misplaced or mislabeled and increases the efficiency and throughput of diagnostic testing to get accurate test results to clients faster.

### BRIEF DESCRIPTION OF THE DRAWINGS

FIG. 1 is a schematic flow chart in accordance with the present invention including physical step sequences and data transfer with a control system for a process of diagnostic testing for specimens including the automated accessioning of specimens with client diagnostic test procedure request data and determination of sufficiency of the specimen for a diagnostic test procedure and the automated sorting of specimens for delivery to specified laboratories based upon the diagnostic test procedure;

FIG. 2 is a schematic illustration of an automated sorting system in accordance with the present invention including transfer stations for automatically directing specimens to selected automated sorters;

FIG. 3 is a perspective view of a system as set in schematically in FIG. 2;

FIG. 4 is a schematic flow chart of a process in accordance with the present invention for determining the sufficiency of a specimen as compared with diagnostic test procedure data stored within memory of a control system or central processing station;

FIG. 5 is a schematic diagram of a software and database architecture for automated sorting of specimens in accordance with an aspect of the present invention;

FIG. 6 is a schematic illustration of a sorting location profile as applied to a plurality of automated sorting stations;

FIG. 7 is a perspective illustration of a preferred carrier combined with a specimen vessels as usable with conveyors for transporting specimens;

FIG. 8 is a schematic illustration of an automated specimen processor in accordance with an aspect of the present invention for determining origin data and specimen attribute data of specimens provided to the specimen processor and for accessioning the specimens along with the central processing station to associate specimens with client diagnostic test procedure request data of the central procession station and to determine sufficiency of specimens for specified diagnostic test procedures;

FIG. 9 is a schematic flow diagram of a portion of automated specimen handling systems and methods in accordance with the present invention showing the receipt of specimens as packaged and unloading of such packaging;

FIG. 10 is a schematic flow diagram of another portion of automated specimen handling systems and methods in accordance with the present invention showing the grouping of specimens on a client basis and the association of specimens with carriers for further transport;

FIG. 11 is another schematic flow diagram of a portion of automated specimen handling systems and methods in accordance with the present invention showing the combination of automated accessioning and sorting of specimens; and

FIG. 12 is a schematic flow diagram of a portion of automated specimen handling systems and methods in accordance with the present invention showing specimens after sorting and the delivery to appropriate testing laboratories.

### DETAILED DESCRIPTION OF PREFERRED EMBODIMENTS OF THE PRESENT INVENTION

The present invention is directed to methods and systems that have applicability for use in diagnostic laboratories, particularly biological specimen diagnostic laboratories that handle large volumes of specimens as are submitted by clients of the diagnostic laboratory for diagnostic testing. Such a laboratory facility, as discussed above in the Background section, may have many associated specific labs, each of which may perform different diagnostic testing based on different technological test procedures. Moreover, in being able to handle esoteric testing of specimens by diagnostic test procedures of a large variety, the specimens must be handled carefully and efficiently and tracked accurately so that the proper diagnostic tests are conducted in a timely manner. In other words, to be effective as an esoteric diagnostic laboratory, a large number of diagnostic test procedures should be available and the laboratory should be able to handle large volumes of specimens for commercial viability.

Efficient and accurate handling of specimens is preferably controlled from the specimen delivery through the completion of the diagnostic test procedure. In addition, efficient and accurate collection and control of data transfer is important in making sure the specimen is handled properly, that the appropriate diagnostic test be conducted, and that the results are accurately reported to the client. Such data collection begins with the placement of a test order from a client for creating a test record, which record may be supplemented with additional specimen information and process control information at any number of points throughout the system process, and is importantly connected with the appropriate designated specimen throughout the process. The present invention is described as particularly directed to the diagnostic testing of biological specimens, such as blood, serum, urine, tissue, and the like while it is understood that the systems and methods of the present invention are applicable where other type components or materials are to be selectively subjected to test procedures and which are submitted for such testing by outside clients. It is further understood that the systems and methods of the present invention are scalable depending on the volume of specimens to the handled through the system and on the number of test procedures that are potentially available to clients. As will be apparent from the following description, scalability can be accomplished by increasing or decreasing the number of similarly utilized components as are functionally described below.

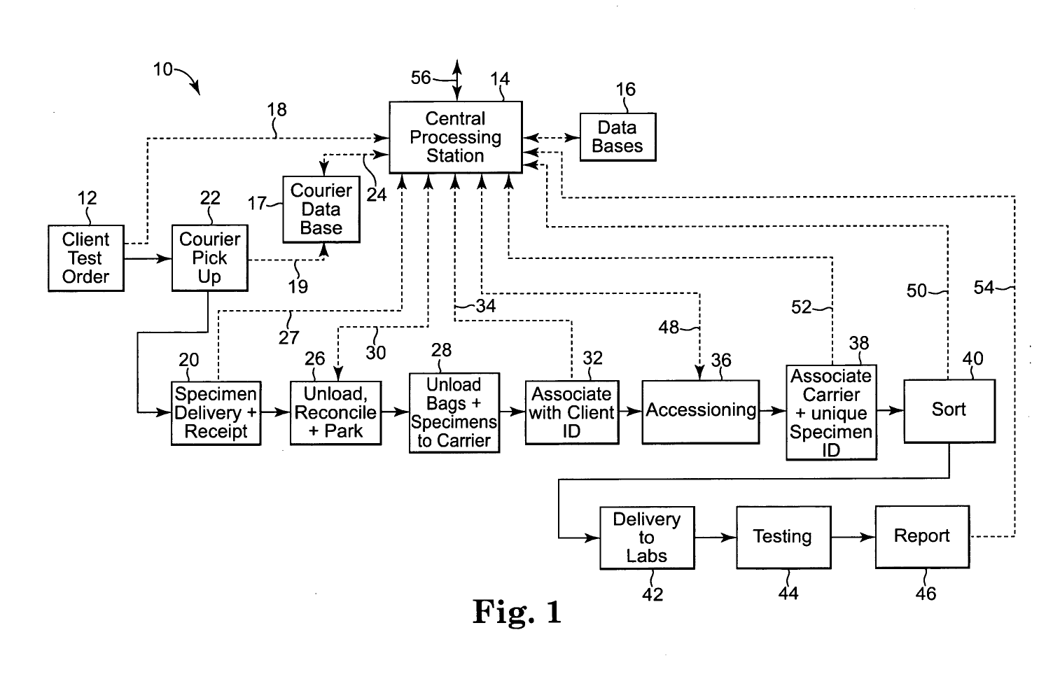

Referring to FIG. 1, a process of diagnostic testing 10 for a specimen is schematically illustrated and that begins with the submission of a client test order shown at box 12 to a diagnostic laboratory. It is noted that throughout FIG. 1, dashed lines are utilized to show the electronic transfer of data as may be facilitated by any public or private network or direct electronic link including the use of phone lines, cable lines, data transfer cables, and the like while the solid lines represent the physical transfer of a specimen from one station to another as are schematically indicated. In each case of data transfer discussed in this application, it is understood and preferable to accommodate two-way data transfer.

In most cases, and as preferable in accordance with the present invention, a client test order is electronically submitted and received by a central processing station 14, which itself preferably comprises one or more data processors and memory of sufficient size and that can more preferably be organized with selectively accessible databases by operating systems and data management software as are conventionally known and commercially available. Moreover, any number of display devices, such as monitors and the like, and input devices, such as keyboards and the like, can be connected with the central processing station 14 in any conventional way. Such a central processing station 14 can comprise any number of computers or servers as may be networked together and as are conventionally available. Box 16 represents any number of specific databases as may be accessed by the central processing station 14. Dashed line 18 represents the submission of a client test order from a client to a diagnostic laboratory having the central processing station 14 for the purpose of receiving the client test order. It is contemplated that such order can be facilitated by any known or developed interface including the use of client software that interacts with application software running on the central processing station 14, by web-based interface tools and software, or the like. Otherwise, the test order may merely comprise an electronic submission of text or code to the central processing station 14 for its extraction of relevant information as may be facilitated by an operator or done automatically. The electronic receipt or input of a client test order preferably begins the creation of a record for each test procedure to be conducted by the lab.

A typical specimen as prepared for delivery comprises a volume of fluid or piece of tissue as would be conventionally contained within a vial or similar vessel and as would include a label or markings indicating relevant information related to the specimen and to the test order information that has been submitted for that specimen. For example, the health-care facility may be designated as well as the physician, patient, and diagnostic test identification information. Such information may be provided in alphanumeric form, whether coded or not, or may be provided in non-readable code, such as a linear or two-dimensional bar-code. Additionally, such a specimen vial or other vessel is typically placed within a sealable bag that itself also preferably includes a unique identifier, such as its own bar-code or the like representing a bag identification number. For temperature control purposes, one or more of such sealed bags may be placed within an insulated container (sometimes called a “styro”) and any number of such specimens may be put into shipping boxes or containers by the courier, as may be done on a client specific basis or as a mix of specimens from multiple clients. Such couriers, whether a private, commercial or governmental delivery service, typically further provide a container tracking identifier, such as an ID number, which is also associated with the identifiers of the specimen bags contained therein and that is electronically transferred as indicated by dashed line 19 to be stored within that courier's internal data base 17 as manifest data for each shipment. Manifest data typically includes the container identifier, the number of bags within that container, the identifiers of each bag, and a client identity for each bag. When packing such shipping containers, each bag identifier is typically scanned to read a bar code or the like from each bag and associated with the shipping container identifier, as also usually provided as a bar code or the like on a container label. The subject courier's internal data base 17 is also typically electronically accessible by the laboratory as indicated by dashed line 24 for shipment tracking (e.g. by tracking numbers or each container) and to view such manifest data (i.e. to view the number of each customers specimen bags that are contained within each identified shipping container).

As indicated in FIG. 1, each physical specimen is to be sent by the client to the diagnostic laboratory, and box 20 represents the delivery and receipt of one or more such specimens at the laboratory facility. Box 22 represents the common and preferable use of a courier as such services are commercially available for picking up any number of specimens from a client, such as at a doctor's office, hospital or other health-care facility. Dedicated couriers normally pick up the specimens from the client and pack the bags containing the specimens into a container for shipping in a manner as described above. Moreover, an electronically accessible courier manifest is preferably created with respect to each container, preferably including data identifying each bag that is provided therein, the number of bags within the container, and the client locations from which the specific bags have been submitted. This information can be very useful in the delivery and receipt of the specimens and for associating the specimens with the test orders received by the central processing station 14. Preferably, the manifest information can be provided to the central processing station 14, such as by an electronic download as indicated by dashed line 24 and as may be facilitated by any known or developed application interface, or any known or developed manner of electronic communication including the use of public and/or private networks. Client test orders as they are received and stored within memory of the central processing station 14 may then be associated with such courier manifest information and together stored within any one or more database 16 that is associated with the central processing station 14 if desired. A preferable use of manifest information to reconcile specimens received with client orders is discussed in greater detail below.

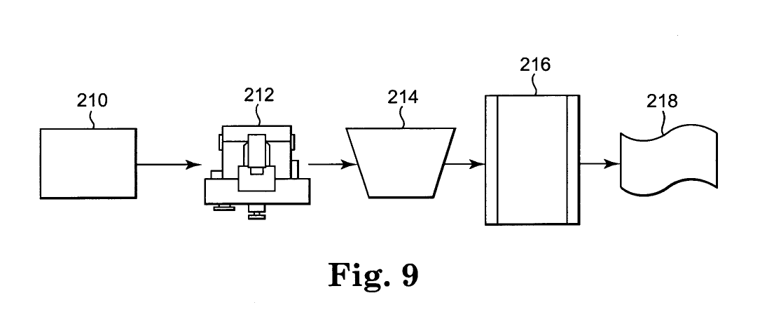

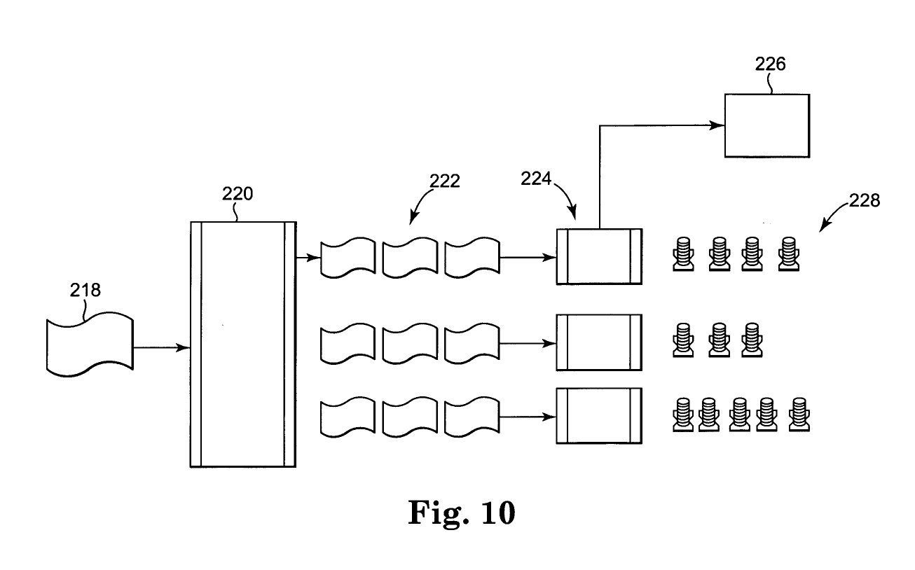

Once one or more containers are received at the diagnostic laboratory facility, the containers are unloaded as indicated at box 26. The unloading process step preferably comprises the unloading of shipping containers and also unloading of any temperature control packaging as is common and the reconciliation of the bags, as determined by the bag identification numbers, with the orders that have been placed by the client and utilizing the courier manifest data, if available. Step 28, as indicated in FIG. 1, represents the further unloading step of removing the specimens as provided in vials or other vessels from the bags and placing the vials or other vessels to the extent possible in carriers to take the specimens further along the system 10 for additional processing steps and as described below. FIGS. 9 and 10 schematically illustrate greater detail of preferred steps that can be conducted as part of the container unloading step 26 and the bag unloading step 28. A scan of the container bar-code by the courier can complete the manifest data showing delivery of the containers as previously defined by the packaging process. Or, a scan by the receiving laboratory can be used to show the receipt of the containers as compared with the manifest data that can be accessed at that time or previously. Dashed line 27 in FIG. 1 represents the electronic transfer of container delivery and receipt at the laboratory to the central processing station 14 whether direct or by way of the courier data base 17. This starts the reconciliation of client orders and received specimens, which reconciliation further preferably includes a comparison with client orders, if received electronically, and the receipt of the actual specimen bags after the unloading process is done. If manifest data is not accessible, reconciliation of client orders can begin with the unloading of the bags and comparison of client orders if received electronically. Otherwise, the unloading and receipt of bags or other containers can begin the creation of an electronic record of a specimen test order from a client where there would be no further need for reconciliation of such order.

In FIG. 9, step 210 shows the receipt of one or more containers, such as boxes, to be unloaded. Step 212 shows a box cutter for opening boxes, which step can be conducted manually or by use of any commercially known or developed box cutting apparatus. After container opening, step 214 indicates the optional handling of temperature control containers and step 216 represents the opening and removal of bags 218 from such temperature control containers, if so provided. As shown in FIG. 10, the bags 218 are preferably provided to a bag processing station 220 where the bags are preferably scanned for bag identification, such as based upon a bag identification bar code or the like, and the electronic transfer of bag identification information to the central processing station 14 is indicated by the dashed line 30. Dashed line 30 may otherwise represent the input of bag identification information manually by an operator to the central processing station 14. As part of the reconciliation process, it is preferable to group specimens on a client by client basis for ease in comparison with client orders as received and with manifest information as available. Based upon expected orders on a client basis, the central processing station 14 can assign physical receiving areas, such as parking lanes or the like of a conveyor system, for grouping all clients orders until the unloading process is complete, or until satisfaction of the receipt of the number of client orders that are expected. Dashed line 30 also thus represents the assignment of grouping areas as transferred to the bag processing station 220, which assignment may be carried out automatically or to inform an operator of each specimen's destination for routing purposes. Each bag scan also allows the central processing station 14 to complete the reconciliation process and to also provide information to an operator or automatic system that a client's order has been successfully received and for subsequent release of the specimen bags for unloading. Step 222 in FIG. 10 represents the sorting of bags on a client basis. During the bag processing, bag reconciliation is preferably performed by the central processing station 14 by correlating bags received (as identified by bag number, for example) with the orders placed and with the specimens as packaged by the courier and indicated by the manifest information. If a specimen is determined missing based upon unmatched test orders to specimens, an attendant could then investigate.

The specimen bags, as are preferably organized on a client basis, are then opened by an attendant, as indicated by step 28 in FIG. 1 and step 224 in FIG. 10. Step 226 represents an optional diversion of unloaded specimens, such as may be done for handling of specimens of odd sizes or types or based upon unreadability of a bag identification number or the like. The specimens as provided within conforming vials or other vessels are preferably then preferably placed within carriers as indicated by step 228.

Referring to FIG. 1, step 32 indicates a preferable association of each specimen carrier with a unique client ID, such as taken from their respective bag identifier, as available in the circumstances where client identifiers are provided in a readable form such as a bar code or the like. Where any order does not contain a unique client ID for each group of specimens, the client ID must be determined from whatever other information or paperwork is provided with the shipment. Step 228 of FIG. 10 preferably also represents the scanning of bag labels and the association of a determined client identifier to each specimen carrier that is used. For any specimen that is received for which client identification cannot be made by scanning or the like from the bag or specimen vial or vessel itself, that specimen can be set aside for manual for the processing by an attendant. That way, even specimens that may arrive without any submission of electronic shipment tracking information or other order information can be processed. The creation of an electronic record can include scanning of any paperwork accompanying the specimen and/or a digital image of the specimen itself. Dashed line 34 indicates the electronic communication of scanned or otherwise entered data to the central processing station 14 as may be an obtained or created within step 32 so as to permit the central processing station 14 to associate the specimen with its client identifier or other created unique identifier for further processing of the specimen.

At this point, it is preferable that each specimen carrier at least be associated with a client identifier as information related to the origin of each specimen. If more information is available or if otherwise desirable, more than the client identifier can be associated with a carrier ID. However, for purposes of the present invention, it is sufficient to have an association between the carriers and client identifiers at this point in the process.

Such specimen carriers may comprise any known or developed carrier, but preferably comprises a carrier provided in the form of a puck that is disk shaped and has the ability to receive and hold a vial or similar vessel in a particular orientation for transfer to further stations of the system. Such a carrier puck 510 and specimen vessel 512 combination is illustrated in FIG. 7. Conveying systems capable of transferring such pucks are conventionally known and commercially available, such as by FlexLink AB of Gothenburg, Sweden. Preferably, each carrier also comprises a unique identifier in the form of an RFID device that is incorporated into the carrier so that when the carrier passes any RFID reader, the specific carrier can be identified. By associating the carrier with the client identifier, client identification of each specimen can be accomplished while the carrier is moving by passing the carrier and specimen past an RFID reader. Association of the carrier and unique client ID is done by an RFID programming device, and the data transfer to and from the central processing station for this purpose is also indicated by dashed line 34. RFID readers and programming devices, themselves, are well-known and conventional.

The remainder of the steps of system 10 as illustrated in FIG. 1 include at box 36, an accessioning step, at box 38 a re-association step, at box 40 a sorting step, at box 42 as step of delivering the specimens to the proper laboratory for diagnostic testing, at box 44 the step of conducting the diagnostic test, and at box 46 the step of storing and reporting the diagnostic test results to the client. The steps of accessioning and sorting the specimens as indicated at boxes 36 and 40 will be discussed in greater detail below. Electronic data transfer between the central processing station 14 and control devices of the accessioning step 36 and sorting step 40 are also facilitated as indicated by dashed lines 48 and 50, respectively, as shown in FIG. 1. Data transfer as may be utilized for the re-association step 38 is indicated by dashed line 52. The act of conducting a diagnostic test procedure of box 44 can be any of those known or developed test procedures based upon any number of known or developed diagnostic technologies as may be conducted on any appropriately provided specimen. As above, a diagnostic laboratory facility would likely include many different labs that may be displaced from one another, and as indicated by box 42 can be delivered to such labs in any conventional or developed way, including manual means, automatic means and using known or development transport manners. As will be apparent after the discussion below, specimens are preferably delivered in trays that are designed to accommodate the type and size of the specimen vessels that are normally used for any particular test procedure. The step of storing and reporting the test results to the client is preferably conducted by electronic means although paper recordation and archival is contemplated. A diagnostic technician or other attendant preferably inputs the results of each diagnostic test into a database of the central procession station 14 with association to the client order and specimen record. Data transfer to the central processing station 14 for the reporting function of step 46 is represented in FIG. 1 by dashed line 54. The results can be electronically provided to the client in a manner similar to that described above for receiving a client order but in reverse or otherwise, as such an electronic data transfer is indicated by arrow 56 in FIG. 1.

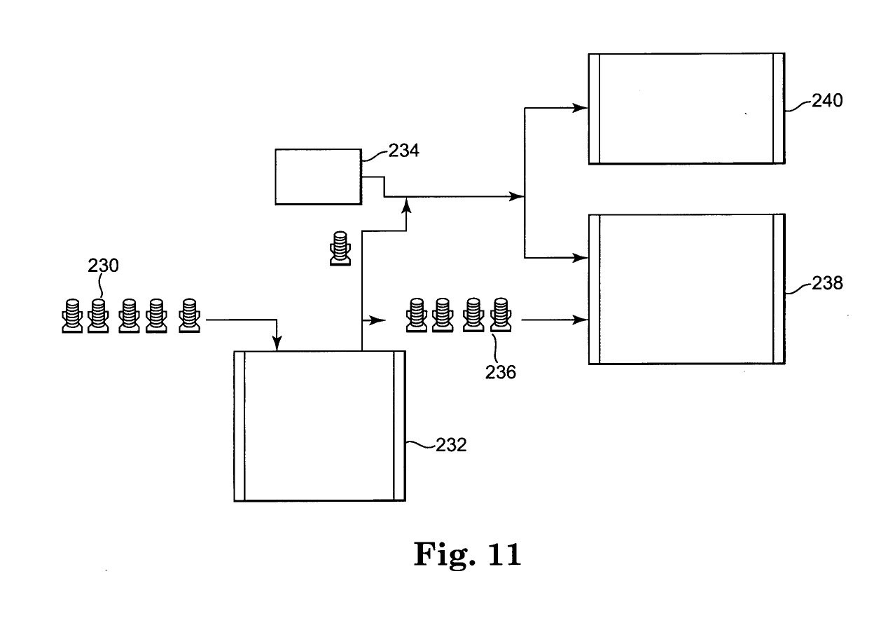

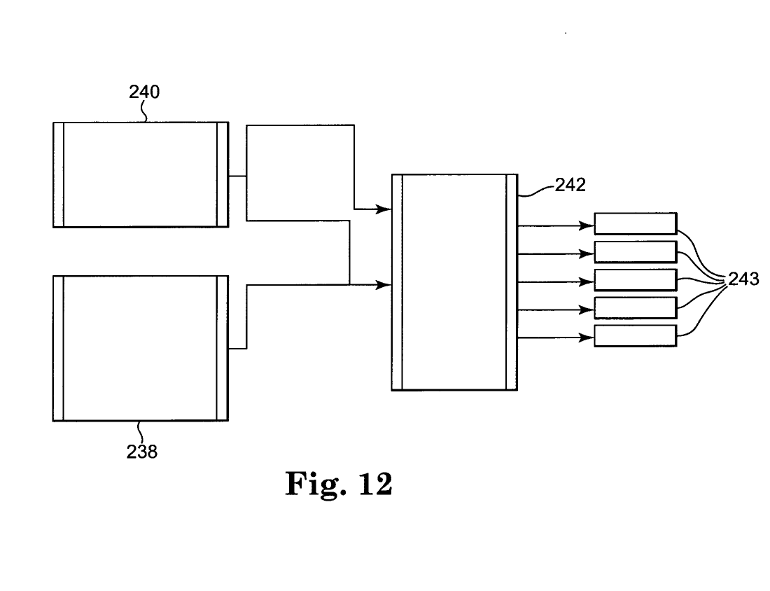

Referring to FIGS. 11 and 12, specimen and carrier combinations 230 are shown initially as leading to one or more specimen processors 232. The specimen processors 232 are described in greater detail below and provide functionality for accessioning the specimens with client orders to make sure that the data record is complete and that a physical specimen is sufficient for the test to be conducted. Any number of such specimen processors 232 can be provided in order to handle what ever volume of specimens are to be handled by the system 10. With the provision of multiple such specimen processors 232, the specimens can be selectively delivered to each specimen processor 232 based upon a balancing the volume of specimens to be handled or on any other criteria of a laboratory. Specimens are preferably directed to any one processor that is determined based upon a pull request manufacturing process. For example, as a specimen processor 232 becomes available to do work, that specimen processor 232 provides a notification (via electronic signal) to the central processing station 14. The central processing station 14 can then preferably assign and then direct all specimens for a specific client to that specimen processor 232 since each specimen can be identified on a client basis by the association of the carriers with client identifiers, RFID reading of the carriers can facilitate this control. Control system 14 can determine to which specimen processor 232 a client is assigned based upon the quantity of specimens known to be present for processing. This allows for a flexible and efficient utilization of any number of multiple specimen processors 232. Box 234 in FIG. 11 represents a manual accessioning step that may be conducted, for example, on specimens that are provided in vessels or otherwise that are not easily handled by the automated equipment of the specimen processor 232. Specimen and carrier combinations 236 are shown as leaving the specimen processor 232, as they are re-associated during step 38 of the schematic of FIG. 1, and as they are then directed to an automated sorting system 238 that is described in greater detail below. The carriers that are re-associated with the specimens may be the same or different as was done in the earlier association step 228. Re-association can be accomplished by a scanning of the unique specimen ID as provided to each specimen during the accessioning process (discussed below) and reading the carrier identifier, as preferably provided as an RFID device within the carrier design. Box 240 represents a manual sorting step as may be applied to specimens that have been manually accessioned in step 234, although manually accessioned specimens may otherwise be handled by the automated sorting system 238 if the manual accessioning provides the specimens in a manner to be successfully handled by the automated sorting system 238. In FIG. 12, specimens are illustrated as leaving either the automated sorting system 238 or the manual sorting step 240 for delivery to any appropriate laboratory for testing. Preferably, one or more pre-analytic stations 242 are also provided as may be variably specified under control of the central processing station 14 for any final preparation of the specimens and to facilitate delivery to any number of labs 243. Any laboratory criteria can be utilized and preferably controlled by way of the central processing station 14 as described in greater detail below for the sorting and delivery process.

The specimen processors 232 can be any functional system or machine having the ability to receive specimens as provided in vials or other determined vessels and as positioned at a determined location within the machine and that is capable of conveying the specimen vessels through one or more identification or analysis stations within the machine and then for selectively transferring each specimen vessels for further processing, such as for sorting and controlled delivery to selective labs, as described below. Such specimen processors 232 may also include further features for verifying specimen physical properties as may be necessary based on a particular test procedure, for holding specimens so that they may be set aside for attendant action, and for aliquoting a specimen for more than one test procedure. A specific example of one machine usable for the specimen processor 232 in accordance with the present invention is the OLA2500 Decapper/Sorter/Archiver/Aliquotter that is commercially available from Olympus America Inc. of Melville, N.Y. In addition to receiving specimen vessels, selectively transferring and conveying them for analysis, and relocating the specimens in trays positioned at predetermined locations, the Olympus OLA2500 machine can transfer a specimen to an aliquoting station for parking the specimen vessel, aspirating any amount or all of the specimen from the vessel, and controllable dispensing the specimen back into one or more new vessels stored in the machine. A preferred specimen processor 232 comprises a modified form of this machine that is fully automated and computer controlled for analysis as may be necessary to determine adequacy of a specimen for a particular test procedure and/or for sufficiency to permit aliquoting for more than one test procedure.

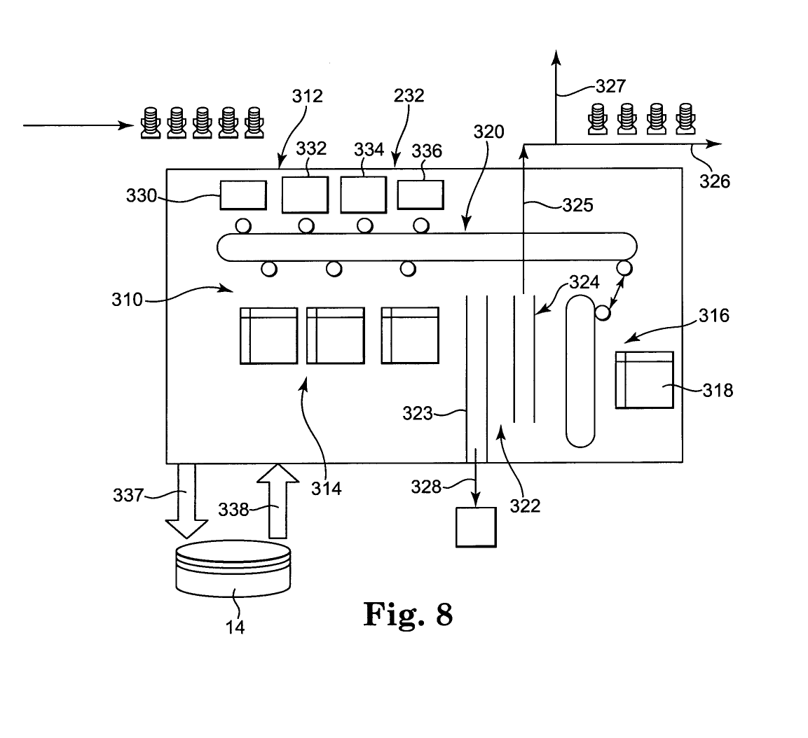

FIG. 8 shows a modified configuration of the Olympus OLA2500 machine, which modifications are preferable for the specimen processing functionality of the present invention. A specimen processing area 310 includes a specimen receiving area 312 and a holding zone 314 for receiving trays in any arrangement with specific locations that can be programmed to receive specimens in a controlled manner. For the specimen processing (or accessioning) function, trays are preferably arranged for providing hold areas for specimens as may be distinct from one another on any criteria, such as by temperature. For example, samples that are held for any reason can be grouped as ambient, refrigerated, or frozen, and the tray or holder can be designed with features for such purpose. Next to that, an aliquoting area 316 is provided including a parking area 318 for holding specimens for any reason and storing new vessels as may be needed for an aliquoting process. An oval shaped conveyor 320 is provided along the back edge of the processing area for moving specimens through one or more analysis zones and to facilitate transfer to a tray within the tray location zone 314. A first transfer robot (not shown) is provided, as are well known and included in the commercial versions of the above-noted machine OLA2500 from Olympus American, Inc., that is movable in the X-Y plane is provided to transfer a specimen from its reception location in a carrier to the conveyor 320. A second transfer robot (not shown) that is also movable in the X-Y plane is provided to transfer specimens from the conveyor 320 to a selected tray location or the aliquoting area 316. Each movement of the first and second transfer robots are programmably controlled. The aliquoting area 316 may also include another transfer robot movable in the X-Y plane to pick up and locate specimen vessels in a location to aspirate and dispense specimen into one or more other vessels. Alternatively, the second transfer robot can move specimens to and from the holding zone 314, conveyor 320 and/or the aliquoting area. Any number of such transfer robots can be used. A label station is also provided in this system for labeling new vessels according to test and other identification information after aliquoting, which labeling function is also programmably controlled. The labeling function is preferably incorporated into the aliquoting area and functions so that every specimen passes through the aliquoting area to at least be relabeled with a unique identifier for further processing.

In order to verify specimen physical attributes or properties to show adequacy of a specimen for one or more selected test procedures, it is preferably to include along the conveying path of the conveyor 320 a scanner 330 for identifying at least client information of the specimen (a bar code reader or the like), a volume measurement device 232 that preferably would utilize an ultrasonic and/or an infra-red sensor to detect the volume level within the vessel and that in conjunction with determining tube size can calculate specimen volume, an electronic imaging device 334, such as a camera, CCD device or other device for the purpose of at least determining specimen color, and a temperature monitor 336, such as comprising one or more non-contact commercially available temperature sensors from Omega Engineering, Inc. of Stamford, Conn. These and any number of other sensors are preferably located along the path of the conveyor 320 so that each specimen can be measured and its information transmitted to the central processing station 14 as indicated by arrow 337. Decisions from the central processing station 14 and relabeling information are provided to the specimen processors 232 as indicated by arrow 338. Electronic information is received, compiled and transferred with each specimen processor 232 by a computerized control system that is operatively connected with each sensor, transfer mechanism, the aliquoting station and the labeling station.

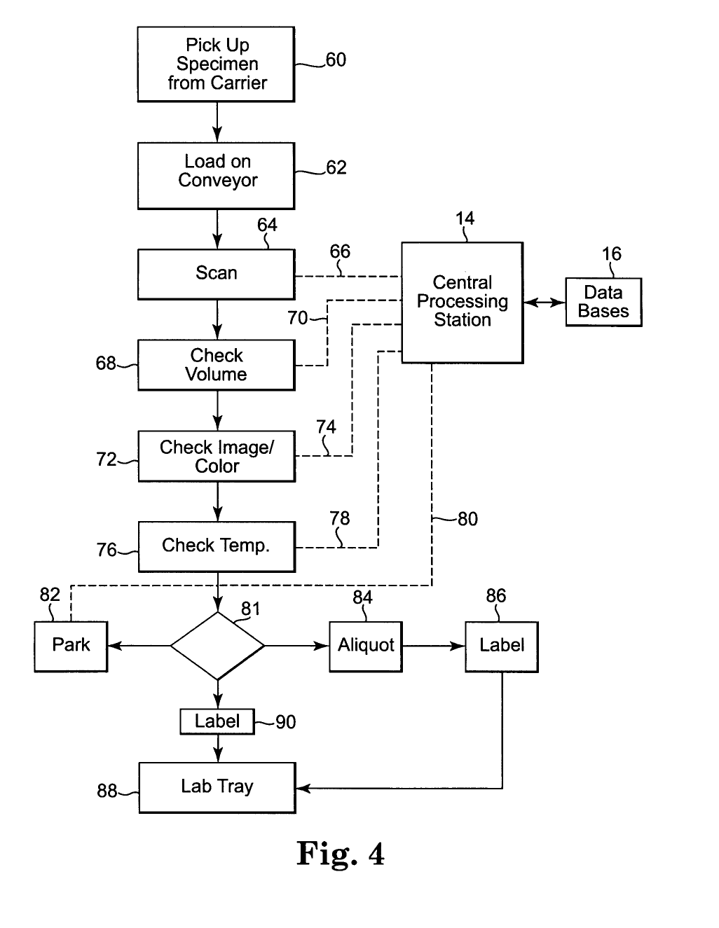

Referring back to FIG. 1, the accessioning step represented by box 36 is made possible by the finctionality of the specimen processors 232 as one or more of such specimen processors 232 are networked to the central processing station 14. Dashed line 48 in FIG. 1 represents the two-way data transfer aspect between the central processing station 14 and each specimen processor 232. The accessioning step 36 and data transfer aspects are illustrated in greater detail in FIG. 4. In particular, the functionality of the specimen processor 232 is important to the validating of each specimen for adequacy of the specimen for the one or more test procedures to be conducted as ordered by a client. Moreover, the physical specimen analysis steps can provide valuable information regarding each specimen for correlation with an ordered test procedure of a client that is associated with the specimen in question, which information may be usable to complete a record of the specimen and test procedure. As above, client diagnostic test procedure request data is stored within memory of the central processing station 14 along with information of diagnostic test procedures themselves, including requirement data to be compared with attribute data as obtained by each of the measuring sensors of the specimen processor 232.

Once a specimen is transferred to the specimen receiving area 312 of a specimen processor 232 it can then be transferred to the conveyor 320. The transfer steps to the specimen receiving area 212 and from that to the conveyor 320 are illustrated in FIG. 4 as boxes 60 and 62. The following information gathering and analysis steps can be conducted in any order and it is contemplated that more or less such steps can be conducted while the specimen is conveyed along conveyor 320 as may be conducted based upon the desired acquisition of relevant information regarding any specimen for any particular application.

Box 64 represents a step of scanning or otherwise reading information (at least client information) from the specimen vessel, such as a bar code scanner, to determine at least origin data of the specimen (e.g. the client) of the specimen and for communication with the central processing station 14 as indicated by dashed line 66.

Box 68 represents a step of determining an attribute of the specimen comprising in particular a volume and/or size of the specimen taking into account the specimen vessel size and type. Dashed line 70 represents a data connection with the central processing station 14 for transferring volume data as attribute data. Based upon diagnostic test procedure requirements or rules as are preferably stored in memory, such as a database 16, and a query from the central processing station 14, a determination can be made as to the sufficiency of the specimen for the test procedure that has been ordered by the client for that particular specimen.

Similarly, box 72 represents a step of determining a specimen color as another specimen attribute, for example from an image, and dashed line 74 represents a data connection with the central processing station 14 for communicating specimen color as attribute data. Again, based upon diagnostic test procedure rules as are preferably stored in memory, such as database 16, and a query from the central processing station 14, a determination can be made to validate whether the specimen is the correct specimen for the test procedure that has been ordered by the client for that particular specimen. As an example, a blood specimen would be expected to be red. If an unexpected specimen image or color is detected, such information may be usable in comparison with other client test orders to check against other yet unvalidated orders. A patient of one client may, for example, have submitted more than one type specimen for various type diagnostic testing. This process thus validates and/or provides data usable in matching specimens and client test orders, which is the goal of accessioning, so that the specimen can be forwarded to an appropriate lab and the diagnostic test can be conducted. The same is true of the volume or size determination in comparing volume of specimen with expected values, but the volume detection also serves to answer the sufficiency of the specimen for one or more test procedures and to potentially permit aliquoting, described more below. As another example, a client may have ordered more than one test procedure that requires a blood specimen. The system of the present invention would preferably expect that either multiple specimens have been sent or that multiple tests are to be conducted from a single specimen. So, until all of that clients specimens have been fully processed, the question may be open. Tracking and validating each specimen through the system will eventually provide the answer, and the specimen in question can be parked in the holding zone 314 (shown in FIG. 8) specimen processor 232 until the determination is made. If multiple specimens are encountered, they can be properly routed after such validation and from wherever they are parked. Or, if only one specimen is found after the client's specimens are fully processed, the specimen can be aliquotted if sufficient specimen volume is present. Rules for the needed volumes for the various test procedures are preferably stored and used from a database 16 of the central processing station 14 and/or as provided with the programmable control of the specimen processor 232.

Box 76 represents a temperature determination station for sensing the specimen temperature as yet another specimen attribute and dashed line 78 represents a data connection with the central processing station 14 for communicating temperature as attribute data. Based upon diagnostic test procedure rules as are preferably stored in memory, such as the database 16, and a query from the central processing station 14, a determination can be made as to the sufficiency of the specimen temperature for the test procedure that has been ordered by the client for that particular specimen. For example, a test procedure may require that the specimen be maintained at a refrigeration temperature to be suitable for such testing. Although the temperature sensing procedure is primarily valuable for determining whether a specimen is adequate for a test procedure, it also provides information that may be usable in validating a specimen with a client order, like the other determinations, in that the rules provide an expectation for a type of specimen, which if not met, may suggest that the specimen is intended for a different test procedure.

Preferably, for using the control system of the specimen processor 232, the network connection 48 with the central processing station 14 can be utilized for providing the diagnostic test procedure requirements or rules as data tables usable from the database memory or as downloaded to the processor or other memory of the specimen processor 232. Also, it is contemplated that each of the communication links 66, 70, 74, 78, 80 and the like can be bundled as a single communication link with the central processing station 14, and/or that such sensor information can be gathered to be transmitted as a single or multiple transmissions to the central processing station 14. Moreover, queries from the central processing station 14 as to sufficiency of any or all of the above noted or other specimen attribute data and/or the origin data can be done after each attribute is measured or after all of the attributes are measured.

As a specimen passes each of the detection stations discussed above within the specimen processor 232, a record is preferably created as may be made part of the record of the specimen and order, as discussed above, and as maintained in the central processing station 14, or may be stored temporarily in any type of memory for the purpose of validating each rule for a given specimen and test procedure as queried so that a decision can be made as indicated by decision step 81. As with certain of the examples noted above, it may be desirable to park a specimen as represented by box 82 until the client's specimens are processed after which the decision step can be made again. If there is no resolution to any outstanding question, the specimen can be located, for example, to a tray that is dedicated for attendant attention and manual further processing. If a decision is made that the specimen is meant to be aliquotted for plural test procedures as represented by box 84, the specimen is transferred to the aliquoting station where the specimen vessel is divided into one or more new specimen vessels with appropriate labeling as indicated at box 86 for location in the predetermined tray as indicated by box 88 and ultimate delivery to the appropriate lab for each desired test procedure. If aliquoting is desirable, but there is insufficient specimen volume for the multiple tests desired by the client orders, the central processing station 14 along with the programmable control system of the specimen processor preferably also provides priority rules (also stored, for example, in a database 16) for determining the test procedures that can be performed from the given specimen information. For example, another determination, such as the temperature determination, may suggest unsuitability of the specimen for one of the test procedures. Or, the specimen may be sufficient for two tests if divided, but otherwise only sufficient for one other test. The rules can prioritize what to do based upon the quantity of test procedures or alternatively based upon which test is more important. Of course, these rules like all of the rules and queries discussed in this application are preferably dynamically controlled through the central processing station 14. If a specimen is determined to be validated with a sufficiently complete record within the central processing station 14, the decision within step 81 would be to route the specimen back out of the specimen processor 232. Box 90 represents the possibility of relabeling any select or all specimen vessels after the decision step 81 and prior to placement on a conveyor to route the specimen for sorting. It is preferable that each specimen be relabeled at this time with a unique specimen ID that will be used throughout the remainder of the sorting, testing and reporting processes.

The sorting process indicated by box 40 in FIG. 1 is preferably conducted using an RFID identification and tracking ability of each carrier that is associated with a specimen (by way of the unique specimen ID now labeled to each specimen), which association is conducted as step 38 by reading both the carrier RFID and specimen unique identifier and associating them within the central processing station 14.

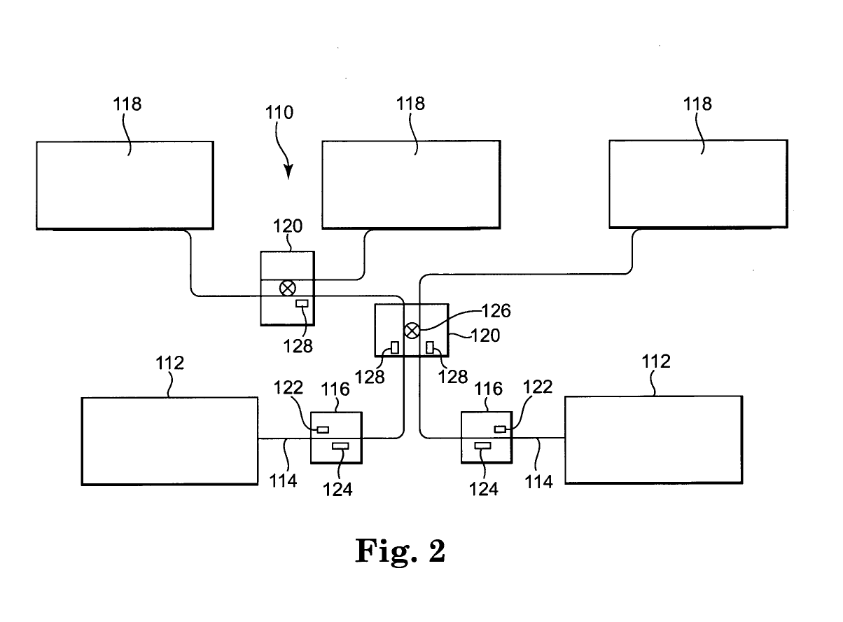

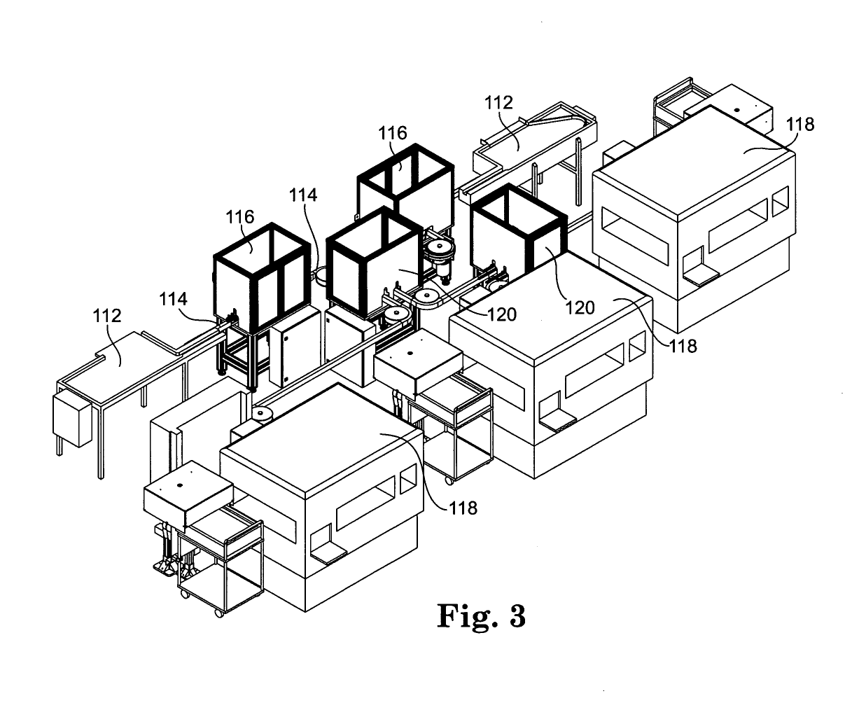

In FIGS. 2 and 3, an automated sorting and transferring system 110 is schematically illustrated. The system 110 is schematically illustrated as comprising a plurality of input stations 112, which may comprise stating areas for specimens and carriers or may comprise the direct feed from any specimen processor 232. The manner of transporting specimens and carriers can be any developed or known conveying system. The specimen and carrier combinations are further transferred by way of a transfer conveyor 114. Stations 116 represent the hardware for associating the carriers and specimens after leaving the specimen processors 232 and for delivery to one or more sort stations 118. Transfer stations 120, of which two are illustrated, selectively transfer and route each specimen to the appropriate sort station 118 as determined for any facility, as discussed in greater detail below. It can be easily seen that any number of sort stations 118 can be provided within the system 110 in accordance with the present invention by utilizing an appropriate number of transfer stations 120. Likewise, any number of input routes or stations 112 can be provided with it being preferable that each input station 112 leads to an associating station 116. The number of each of these components can be chosen based on the volume of specimens to be processed through the system 110 or to facilitate other efficiencies, such as may be based on client specific requirements or diagnostic laboratory needs.

As a specimen and carrier combination travels along conveyor 114 from the specimen processors 232, but before any transfer station 120, the association of the specimens unique identifier and carrier identification is done. In particular, the specimen unique identifier provided on the specimen label as a bar-code or the like and the carrier identification RFID device or the like are preferably each read, such as by a bar-code reader 122 and an RFID reader 124 as illustrated along the conveyor path 114 as station 116. Other type readers or combinations thereof are contemplated based upon the identification means utilized by the specimen and carrier. The specimen and carrier can be identified in either order or at the same time. As indicated by the dashed line 52 in FIG. 1, the association of the specimen and carrier at this point can be electronically transferred to the central processing station 14 for tracking purposes.

Transfer stations 120 each preferably include a transfer mechanism 126 for controllably transferring any specimen from one conveyor segment to another to control the ultimate delivery of each specimen to the proper sort station 118 under whatever criteria is applied at any given time. Sort stations 116 are schematically illustrated collectively in FIG. 12 as the automated sorting system 238. A transfer mechanism 126 may be provided as an electronic motor driven slide type device, such as those commercially available from Intelligent Actuators Inc. of Torrance, Calif. In order to accurately identify each specimen as it is positioned within each transfer station 120, a carrier reader 128, such as an RFID reader, is preferably positioned and synchronized to the transfer mechanism 126 for identifying each specimen (based upon the association of the carrier identification and specimen identification) and selectively activating the transfer mechanism 126 to deliver each specimen to the appropriate conveyor segment that will lead to the select sort station 118.

The dashed line 50 in FIG. 1 represents in another aspect the ability to control the transfer stations 120 so that any determined criteria of the diagnostic laboratory facility can be set within the central processing station 14 for utilizing the sort stations 118. Preferably, specimens are delivered to one or another of the sort stations 118 based upon the labs to which the specimens are to be delivered. For example, each sort station 118 may be dedicated to a single lab or to any plurality of labs as may be useful for processing the batch of specimens through the system 110. The determined labs for each sort station 118 can be changed at any time as may be appropriate based upon an analysis of the types of client test orders received, the locations of the various labs with respect to one another, the delivery schedules of each respective lab, and the like.

Sort stations 118 provide the functionality of sorting the specimens for delivery to the correct laboratory location and advantageously controls specimen flow based on laboratory schedules and batch sizes. A sort station 118 can comprise a machine such as the Olympus OLA2500 machine described above with respect to specimen processors 232 as illustrated in FIG. 8. Sorters 118, however, would not require the aliquoting function station. Specimens are identified by reading the carrier's RFID prior to transferring the specimens to the conveyor 320 or by reading specimen information (the assigned specimen identifier on the label) by a reader, such as at 330. A transfer robot (not shown) moves each specimen to a specifically assigned and located tray within the processing area 314. Trays are assigned locations by the control of the sorter 118 or central processing station 14 to receive specimens destined for a particular lab. Specimens are preferably routed to a designated sorter 118 as assigned by the central processing station 14 based on laboratory schedules, batch sizes and the volume of specimens determined to be routed for each test procedure.

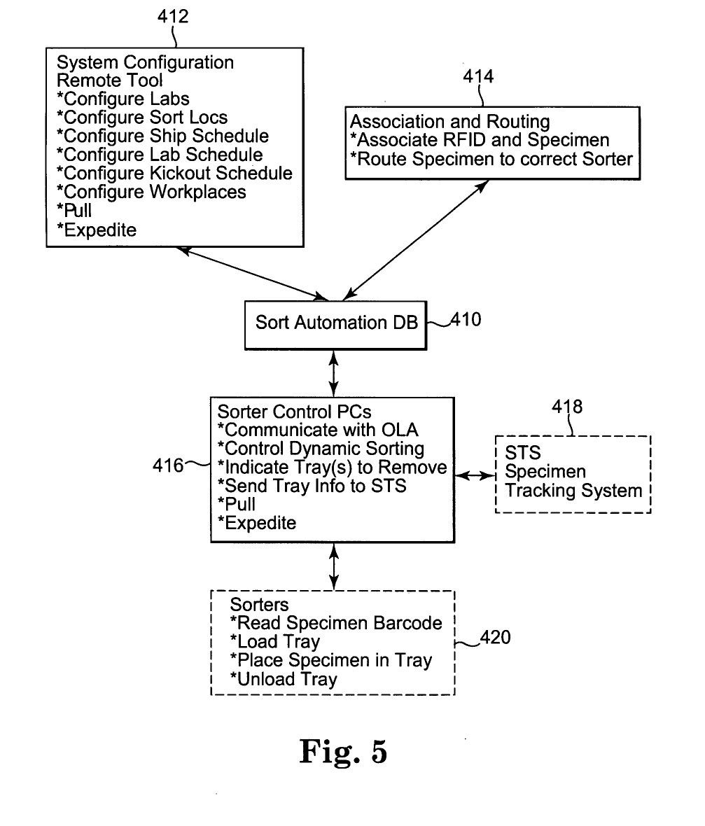

A basic control software block diagram is set out in FIG. 5 showing the basic architecture for the sorting aspect of the present invention and as such communicates with a specimen tracking system that receives the many data input actions noted above in the discussion of how the central processing station 14 interacts with and tracks specimens throughout systems in accordance with the present invention and creates records with respect to each specimen and client test order. The sort control software is preferably resident within the central processing station 14 as may comprise any number of computers, servers and the like with appropriate input and display devices.

The software architecture for the sorting function preferably includes those elements illustrated with a solid line border including a sort automation database 410, system configuration files 412, association and routing data files 414 and control software 416 for dynamically controlling the sorting function and providing control data to the sort stations 118 with respect to tray positioning, timing aspects for tray removal, or other commands as may be necessary to control desirable specimen routing and attention. Box 418 represents the specimen tracking software (STS) as such interfaces with control software 416 and box 420 represents the operational software of the sort stations 118 as such interfaces with the control software 416. As noted above, the operation software of the specimen processors 232 and sorters 118 preferably comprises the software provided by a supplier of the specimen processors, such as that of Olympus America Inc. within its OLA2500 equipment as such is provided with a common interface protocol for exchanging data and control signals with the control software 416. Preferably, however, all decision making is preformed by the central processing station 14 and its control software 416. In each of the data transfer communication links noted on FIG. 5, as with preferably all data links discussed in this application, it is preferable that such links permit two-way data transfer. The specimen processor software 420 preferably includes the ability to read specimen barcodes or other identifier, to control the proper selective tray loading, to control the proper specimen loading in a predetermined tray, and to unload a tray at an appropriate time or event, which timing and/or other event occurrences are based upon the data in the system configuration files.

The sort automation database 410 is the main database to communicate with the control software 416, which database 410 can comprise any number of database structures and database operation control software, such as the SEQUEL (SQL) database management software commercially available from Microsoft Inc. of Redmond, Wash. The system configuration files 412 can include any number of files related to how the sort operation is to be dynamically controlled by providing the information files needed to define sort modes including aspects of the number and type of labs, locations of them, shipping schedules, lab schedules, and workplace data, the purpose of which is to provide direction as to how many specimens, sorted to what level of granularity (for example this system may be able to sort vials to different predefined groupings within each lab), should be delivered and when to each associated laboratory. The association and routing files 414 preferably comprises at least a data table of the association of the carrier identifiers (preferably by RFID) and their lab destination. These data tables allow for each lab destination to be assigned to a specific sorter 118 so that each specimen destined for that label will be placed in a try from only one of the sort machines. For example, the specimen destined for immunoassay testing may be designated to one sorting machine 118. Then, all other specimens destined for that same lab location will also be directed to the same sorter 118. This design allows for the destinations to be changed as needed based on the information in the database referenced above.

An important aspect of the control software 416 is to provide appropriate signals to the transfer stations 120, discussed above, based upon an assignment of the sort stations 118 for any determined criteria of the diagnostic laboratory. Moreover, communication of the control software 416 with the STS software 418 and the software 420 of the specimen processors 232 allows for an electronic tracking record for the location of the specimen as it proceeds through the process.

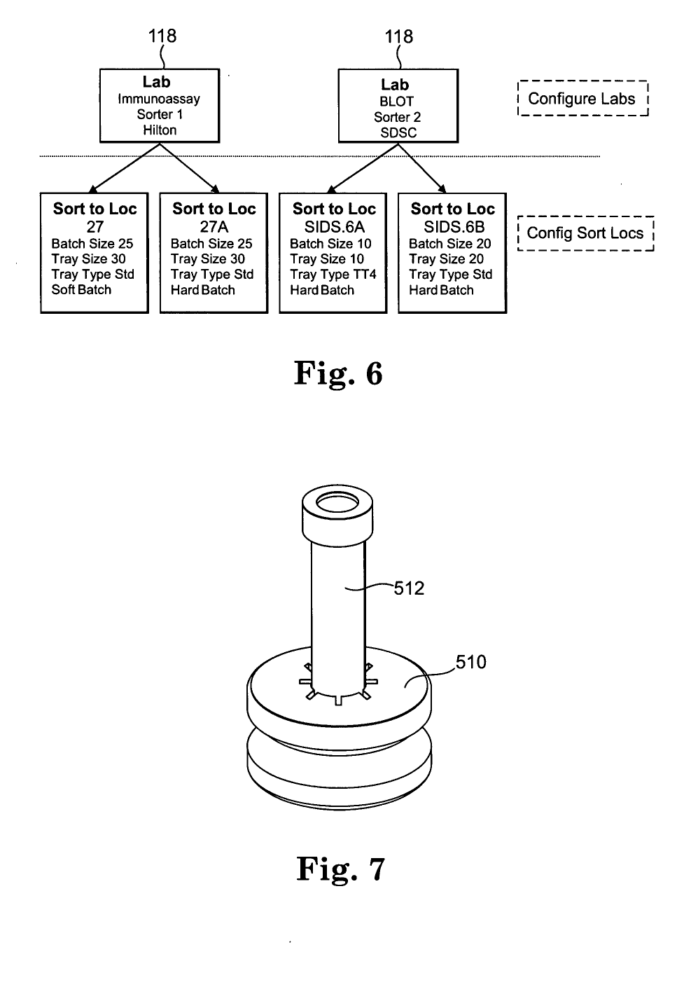

FIG. 6 illustrates an example of a sorting location profile as applied to a plurality of sort stations 118. Each sort stations 118 preferably is provided with a unique identifier, and each more preferably is predetermined to handle and process specimens as directed to a certain technological area, for example immunoassay, or the like, and as provided for testing to one or more specifically designated labs. For each sort station 118, a plurality (two illustrated for each) of sort-to locations 119 are determined as are defined preferably by the location identifier of a designated zone of the tray location zone 214, discussed above, the batch size for processing, the tray size and type, and any other relevant batch or tray related information. The plurality of sort-to locations 119 for each sort station 118 represents any of the different locations that will be directed to the sort station 118 and placed into trays that can be delivered to the labs.

1. A method of automated sorting of specimens provided for diagnostic testing comprising:
assigning one of a plurality of automated sorters to receive specimens on the basis of at least one of a diagnostic test procedure to be performed on the specimens, a particular laboratory to perform the diagnostic test procedure, and a schedule of a particular laboratory;
designating the assignment of an automated sorter to a first transfer station having the ability to transfer a specimen to a plurality of automated sorters;
conveying a plurality of specimens to the first transfer station;
determining identification information of a first specimen and directing the first specimen for delivery to the assigned one of the plurality of automated sorters based upon the designation of assignment to the first transfer station;
conveying the first specimen to the assigned one of the plurality of automated sorters; and
automatically sorting specimens including the first specimen by the assigned one of the plurality of automated sorters on the basis of predetermined destinations for specimens.

2. The method of claim 1, including the steps of assigning a plurality of automated sorters to receive specimens on the basis of at least one of a diagnostic test procedure to be performed on the specimens, a particular laboratory to perform the diagnostic test procedure, and a schedule of a particular laboratory, and conveying at least a second specimen to the first transfer station, determining identification information of the second specimen and directing the second specimen to a different automatic sorter than the first specimen.

3. The method of claim 2, further comprising the step of assigning the same automated sorters to receive specimen to a second transfer station and conveying a plurality of specimens to the second transfer station, and as a result of determining identification information of the plurality of specimens, direction specimens to the plurality of automated sorters based upon the assignments.

4. The method of claim 2, wherein the specimens are conveyed to and from the first transfer station as removably supported by carriers, the carriers including an RFID device to uniquely identify the carrier.

5. The method of claim 4, further comprising the step of associating each carrier with a previously assigned unique specimen identifier of a specimen before the step of determining identification information, which step comprises reading the RFID device of the carrier for the purpose of identifying the specimen.

6. A method of automated accessioning of specimens combined with the automated sorting of the specimens as set out in claim 5, further comprising the following steps conducted prior to conveying specimens to the first transfer station:
determining information related to the origin of a first specimen from the first specimen and transferring first specimen origin data to a control system;
measuring at least one attribute of the first specimen and transferring first specimen attribute data to the control system;
comparing diagnostic test procedure request data stored within memory of the control system to the first specimen origin data and attribute data; and
associating the first specimen with a specified diagnostic test procedure stored within the memory of the control system.

7. The method of claim 6, further comprising the step of assigning a unique specimen identifier to the first specimen during the automated accessioning after associating the first specimen with a diagnostic test procedure so that the control system thereafter associates the unique specimen identifier with a diagnostic test procedure.

8. A method of automated accessioning of specimens for diagnostic testing comprising:
determining information related to the origin of a first specimen from the first specimen and transferring first specimen origin data to a control system;
measuring at least one attribute of the first specimen and transferring first specimen attribute data to the control system;
comparing diagnostic test procedure request data stored within memory of the control system to the first specimen origin data and attribute data; and
associating the first specimen with a specified diagnostic test procedure stored within the memory of the control system.

9. The method of claim 8, further comprising a step of determining whether the first specimen is sufficient for the specified diagnostic test procedure based upon the first specimen attribute data as compared with diagnostic test requirement information also stored within memory of the control system and associated with the specified diagnostic test procedure.

10. The method of claim 9, wherein the step of associating the first specimen with a specified diagnostic procedure comprises associating the first specimen with a plurality of specified diagnostic procedures and attribute date is used to determine sufficiency of the sample for the plurality of specified test procedures.

11. The method of claim 10, further including the step of aliquoting a volume of the first specimen into a plurality of vessels for the plurality of specified diagnostic test procedures.

12. The method of claim 9, further including the steps of measuring a plurality of attributes of the first specimen and transferring measurement data to the control system, the measuring steps comprising determinations of a plurality of specimen temperature, volume, and color by utilization of automatic sensors having output to the control system.

13. The method of claim 9, further including the step of storing diagnostic test procedure request data within memory of the control system based upon submissions of client requests.

14. The method of claim 13, further including the steps of assigning a client identifier to the first specimen after receipt of the first specimen, positioning the first specimen within a carrier having RFID device with a unique carrier identifier, and associating the client identifier with the unique carrier identifier of the carrier.

15. The method of claim 14, wherein the step of determining information related to the origin of the first specimen comprises identifying the client identifier from the RFID device of the carrier for the first specimen.

16. The method of claim 15, further including the step of temporarily holding the first specimen while accessioning other specimens including other specimens having the same client identifier as the first specimen, and after associating at least a second specimen with the same client identifier with s specified diagnostic test procedure, associating the first specimen with the specified diagnostic test procedure based in part on the elimination of the second specimen as compared with a client's diagnostic test procedure request data.

17. The method of claim 16, further comprising the step of rejecting a specimen as being insufficient for the specified diagnostic test procedure.

18. A system for automated sorting of specimens provided for diagnostic testing comprising:
a plurality of automated sorters for automatically sorting specimens by selectively transferring specimens to predetermined destinations of the sorter;
a transfer station for determining identification information of specimens and directing the specimens for delivery to an assigned one of the plurality of automated sorters based upon a specimen type assignment provided to the first transfer station;
a control system for assigning at least one of a plurality of automated sorters to receive specimens on the basis of at least one of a diagnostic test procedure to be performed on the specimens, a particular laboratory to perform the diagnostic test procedure, and a schedule of a particular laboratory, and for transferring data indicating the specimen type assignment to the transfer station; and
conveying means for selectively transferring specimens from the transfer station to the automated sorters.

19. A system for automated accessioning of specimens for diagnostic testing comprising:
a specimen processor including means for determining information related to the origin of a specimen from the specimen, means for measuring at least one attribute of a specimen and means for transferring specimen origin data and specimen attribute data to a control system;
the control system including a data processor and having a plurality of diagnostic test procedural requirements stored within memory of the control system, the control system further include programming for receiving and storing data related to diagnostic test procedure requests, and for comparing specimen origin data and attribute data received from the specimen processor to diagnostic test procedure requests when stored in memory of the control system so as to associate a specimen with a specified diagnostic test procedure stored within the memory of the control system.
